(19) United States
(12) Patent Application Publication
(10) Pub. No.: US 2023/0381386 A1
(43) Pub. Date: Nov. 30, 2023

(54) HEMODIALYSIS FILTER ROTATOR

(71) Applicant: The University of Vermont and State Agricultural College, Burlington, VT (US)

(72) Inventors: Macaulay ONUIGBO, Burlington, VT (US); Adam LOCKE, Waterville, VT (US)

(21) Appl. No.: 18/250,092

(22) PCT Filed: Oct. 22, 2021
(86) PCT No.: PCT/US2021/056146

§ 371 Date: Apr. 21, 2023

Related U.S. Application Data
(60) Provisional application No. 63/094,958, filed on Oct. 22, 2020.

(51) Int. Cl. A61M 1/16

(52) U.S. Cl. CPC A61M 1/1652

(57) ABSTRACT

The present invention has to do with a method and system rotating a hemodialysis filter to provide anticoagulation drag free hemodialysis. Included is a programmable motor controller: a hemodialysis filter holder; and a rotating motor connected to the hemodialysis filter holder, wherein the programmable motor controller rotating motor rotates the hemodialysis filter holder according to a predetermined program.

### CROSS-REFERENCE TO RELATED APPLICATIONS

The present application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith:
U.S. provisional patent application 63/094,958 entitled “Hemodialysis Filter Rotator”, naming Macaulay Onuigbo as first inventor, filed 22 Oct. 2020.

### BACKGROUND

### 1. Field of Use

The present invention relates to a hemodialysis filter and more particularly, the present invention relates to a hemodialysis filter rotator.

### 2. Description of Prior Art (Background)

A hemodialysis device is generally used to clean the blood of patients who have a reduced kidney function or end stage renal disease (ESRD). The dialysis device principally consists of a filter consisting of a semi-permeable membrane, on one side of which passes blood and on the other side of which passes a dialysis solution. By designing the composition of the dialysis solution in a specific manner, an exchange of electrolytes across the semi-permeable membrane and reconditioning of the blood can thus take place.

Dialysis filters often clot and require extra saline flushes and/or anticoagulation drugs such as HEPARIN to prevent clotting. However, Heparin-free hemodialysis is usually obligatory in immediate post-operative states, bleeding diathesis, and in critically ill patients. Thus, there is a need to prevent clotting without extra saline flushes and/or anticoagulation drugs such as HEPARIN.

### BRIEF SUMMARY

The foregoing and other problems are overcome, and other advantages are realized, in accordance with the presently preferred embodiments of these teachings.

A hemodialysis filter rotator is provided. Included is a programmable motor controller; a hemodialysis filter holder; and a rotating motor connected to the hemodialysis filter holder, wherein the programmable motor controller rotates the hemodialysis filter holder according to a predetermined program.

The rotating motor rotates a hemodialysis filter, such as a CAR-170-C NXSTAGE Chronic Cartridge Hemodialysis Filter, for example. The programmable rotation of the filter prevents clotting of the hemodialysis extracorporeal system. Thus, enabling the consistent hemodialysis treatments whether at home or in the hospital without the need for anticoagulation with drugs such as HEPARIN; enabling the patient with end stage renal disease to undergo HEPARIN-free hemodialysis whether at home or in the hospital. By avoiding the use of HEPARIN, hemodialysis can be more safely carried out in very sick patients, in patients with bleeding diathesis, in patients immediately following surgical operations and in patients where the use of HEPARIN is contraindicated. Furthermore, the avoidance of HEPARIN results in cost savings and the avoidance of any other potential adverse effects of HEPARIN such as bleeding, thrombocytopenia, and other hypersensitivity reactions.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a pictorial illustration of a hemodialysis device with rotating motor in accordance with the present invention;

FIG. 1A-FIG. 1D is a pictorial illustration of one filter rotation schedule in accordance with the present invention;

FIG. 2 is a table showing a case study transition to anticoagulation-free hemodialysis in mid-December 2019 with no more need for repeated normal-saline flushes; and

FIG. 3 is a table showing the advantages of the present invention's HEPARIN free hemodialysis.

### DETAILED DESCRIPTION

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example; and

If the specification states a component or feature “may,” “can,” “could,” “should,” “preferably,” “possibly,” “typically,” “optionally,” “for example,” or “might” (or other such language) be included or have a characteristic, that particular component or feature is not required to be included or to have the characteristic.

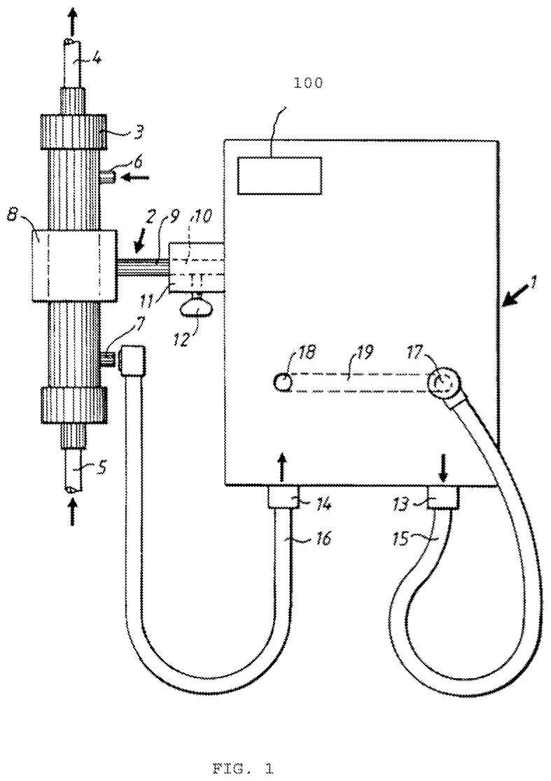

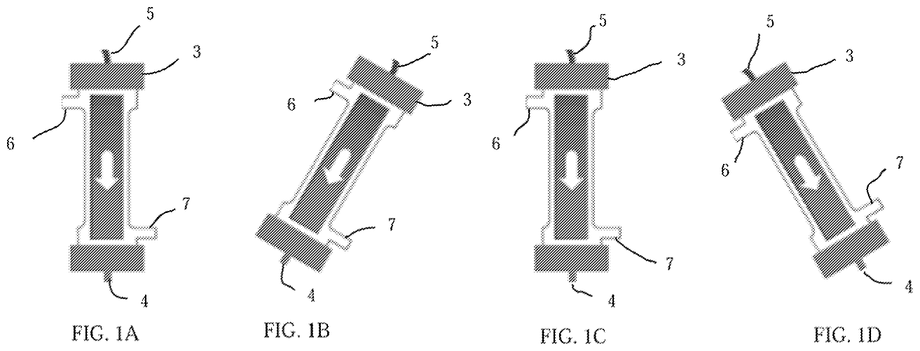

Referring to FIG. 1, shows the lower left-hand portion of a dialysis machine 1. On its front face, the dialysis machine 1 comprises a holder 2 for a dialysis filter 3, such as a hollow-fiber dialysis having a substantially circular cross-section. One such example may be a CAR-170-C NXSTAGE Chronic Cartridge Hemodialysis Filter. The interior space of the hollow fibers are connected by means of tubes 4 and 5, to the patient or, during priming, with a source of sterile priming solution, such as physiological common salt solution. The exterior space outside the hollow fibers is enclosed by a cylindrical tube. This exterior space is connected to coupling arrangements in the form of two nipples, 6 and 7, for connection to the circuit within the dialysis machine for dialysis solution.

The holder 2 comprises an engagement device 8 which cooperates with, and holds, the dialysis filter approximately at its middle. The engagement device 8 is provided with a rod 9 which is inserted into a horizontal hole 10 in a miniaturized motor 11 in the side of the dialysis machine. The rod 9 is maintained within the hole 10 with the aid of a screw 12.

The dialysis machine 1 is provided with a drain 13 for dialysate solution and an inlet 14 for dialysate solution. The outlet 13 and the inlet 14 are provided with tubes, 15 and 16, which, in the stand-by position, are coupled to separate bypass couplings, 17, 18 and 19, positioned on the front of the dialysis machine. FIG. 1 shows the tube 15 from the outlet 13 coupled to the bypass coupling 17. The tube 16 is shown disconnected from the bypass coupling 18 and about to be attached to the lower nipple 7 of the dialysis filter, that lower nipple functioning as an outlet from the dialysis filter. The tube 15 is then disconnected from the coupling 17 and attached to the upper nipple 6 of the dialysis filter. Tubes 4 and 5 are coupled to the sterile priming solution. The dialysis filter 3 is then ready for priming.

For priming, sterile physiological common salt solution is fed through the dialysis filter from below by means of the tube 5 and out through tube 4 so that all of the air is forced out from the spaces within the hollow fibers. At the same time, dialysate solution is supplied through inlet 6 and out through the outlet 7 in countercurrent flow.

Still referring to FIG. 1, miniaturized electric motor 11, controlled by programmable motor controller 100, rotates the dialysis filter 3 to-and-fro, back-and-forth, clockwise, then counterclockwise. Referring also to FIG. 1A-FIG. 1D, the dialysis filter 3 can be rotated from the 12 o'clock position, FIG. 1A clockwise to the 2 o'clock position FIG. 1B, it remains there for 15 minutes, then the filter is rotated counterclockwise to the 12 o'clock position FIG. 1C, and it remains again here for 15 minutes, and then it is again rotated further counterclockwise to the 10 o'clock position FIG. 1D, it stays there for 15 minutes, and then the filter is rotated clockwise back to 12 o'clock position FIG. 1A where it is allowed to stay for 15 minutes. This to-and-fro, back-and-forth, clockwise, then counterclockwise alternating rotational movements are then continued throughout the duration of the hemodialysis treatment as the blood continues to go around in the extracorporeal circuit and all the essential elements of hemodialysis are accomplished for the patient. It will be appreciated that the times and positions are exemplary and the filter 3 may be rotated to any suitable position for any suitable time determined by the programmable motor controller 100. It will also be appreciated that rotating filter 3 to different positions for predetermined times reduces filtered material buildup in any one area of the filter 3 and also reduces filter blood clots.

### Case Report

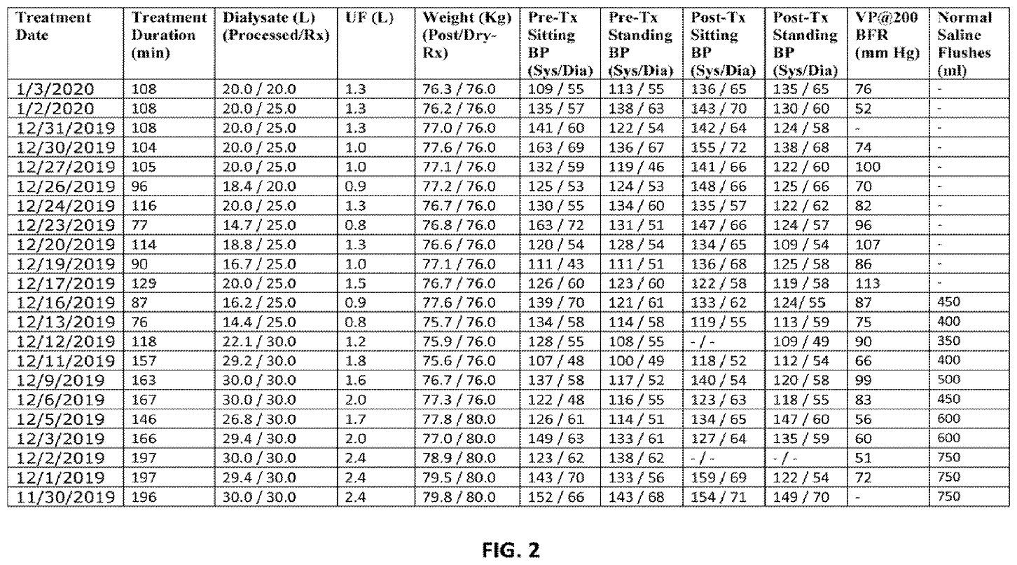

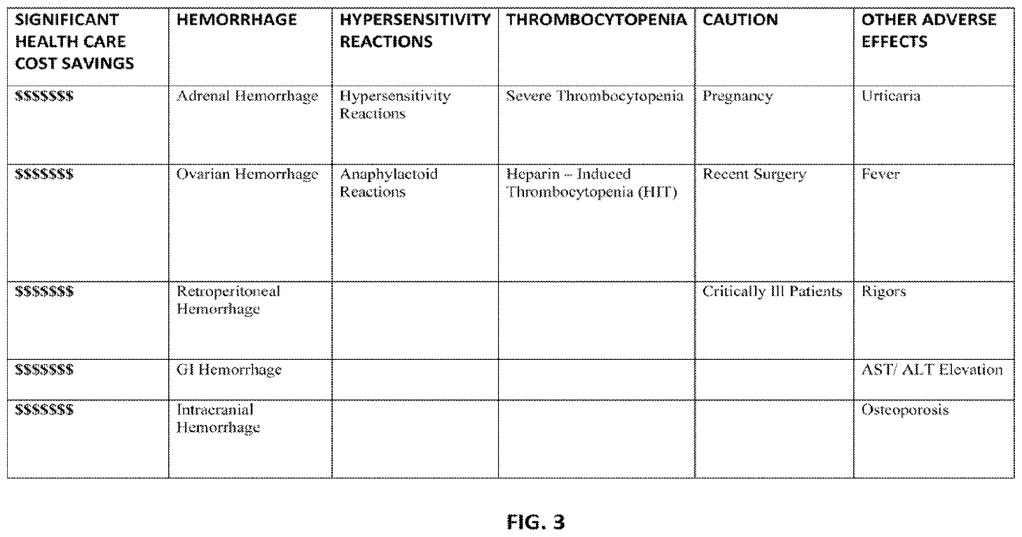

An 87-yo white male with ESRD and atrial fibrillation on Coumadin, on maintenance Home Hemodialysis (HHD) with a NxStage machine, underwent laparoscopic appendicectomy. This was complicated by intra-abdominal abscess, sepsis, and tamponade from a bloody pericardial effusion. He needed emergent therapeutic pericardiocentesis. Coumadin was promptly discontinued. He was discharged home on HEPARIN-free HHD. HHD was complicated by recurrent clotting of the extracorporeal system despite massive quantities of saline flushes. It was observed that the horizontally placed CAR-170-C NXSTAGE Chronic cartridge (hemodialysis filter) exhibited early “clot” formation at the 12 o'clock spot, despite the repeated large-volume normal saline flushes. It was observed that blood was pooling around this site and that the first signs of clotting was appearing here. Through multiple and staggered physical manipulations and maneuvers of the HD filter, it was collaboratively confirmed: Rotating the horizontally placed HD filter along its long axis, 60 degrees clockwise ×15 minutes, return to the neutral position ×15 minutes, followed by rotating the filter another 60 degrees counterclockwise ×15 minutes, and then a return to the neutral position ×15 minutes had promptly and consistently resolved the clotting problem. These rotating steps are simultaneously continued throughout the HHD treatment. The extracorporeal system and HD filter had stopped clotting and for over seven months now, the patient has not needed normal saline flushes for smooth anticoagulation-free HHD. This transition occurred in December 2019 and since January 2020, the HHD runs have been generally smooth and uneventful (FIG. 2). The advantages of anticoagulation-free hemodialysis without the use of Heparin are several including cost-savings and the avoidance of drug-related adverse effects. These advantages are shown in FIG. 3

It will be appreciated that the to-and-fro, back-and-forth, clockwise, then counterclockwise alternating rotational movements of the horizontally placed hemodialysis filter produced antigravity effects that it was able to maintain fluidity of the blood in the hemodialysis filter 3 capillaries thus enabling consistent and successful completion of anticoagulation-free frequent home hemodialysis in subject patient over the past 10 months at a frequency of 4 hemodialysis treatments a week. The anti-gravity effects along the long axis of the cartridge, provide physical defense against progressive clotting of the filter fibers.

It should be understood that the foregoing description is only illustrative of the invention. Thus, various alternatives and modifications can be devised by those skilled in the art without departing from the invention. Accordingly, the present invention is intended to embrace all such alternatives, modifications and variances that fall within the scope of the appended claims.

What is claimed is:

1. A hemodialysis filter rotator comprising:
a hemodialysis filter holder; and
a rotator connected to the hemodialysis filter holder.

2. The hemodialysis filter rotator as in claim 1 further comprising a programmable motor controller, wherein the programmable motor controller rotating motor rotates and holds the hemodialysis filter holder at a predetermined angle according to a predetermined program schedule.

3. The hemodialysis filter rotator as in claim 2 wherein predetermined program schedule comprises:
rotating the hemodialysis filter holder from a 12:00 o'clock position to a 2 o'clock position for 15 minutes;
rotating the hemodialysis filter holder from the 2 o'clock position to the 12 o'clock position for 15 minutes;
rotating the hemodialysis filter holder from the 12:00 o'clock position to a 10 o'clock position for 15 minutes; and
rotating the hemodialysis filter holder from the 10 o'clock position to the 12 o'clock position for 15 minutes.

4. A hemodialysis system, the system comprising:
a rotatable hemodialysis filter holder;
a rotator connected to the hemodialysis filter holder; and
wherein the hemodialysis system does not comprise a Heparin input device.

5. The hemodialysis system as in claim 4 further comprising a programmable motor controller, wherein the programmable motor controller rotating motor rotates and holds the hemodialysis filter holder at a predetermined angle according to a predetermined program schedule.

6. The hemodialysis system as in claim 5 wherein the hemodialysis filter holder comprises a hemodialysis filter.
